(19) United States
(12) Patent Application Publication
(10) Pub. No.: US 2020/0261356 A1
(43) Pub. Date: Aug. 20, 2020

(54) Broad-spectrum nano biocide

(71) Applicants: Max Galland, New York, NY (US); Chloe' Galland, New York, NY (US)

(72) Inventors: Max Galland, New York, NY (US); Chloe' Galland, New York, NY (US)

(21) Appl. No.: 16/873,007

(22) Filed: Jan. 15, 2020

Related U.S. Application Data
(60) Provisional application No. 62/796,986, filed on Jan. 25, 2019.

(51) Int. Cl. A61K 9/00; A61K 33/242; A61K 33/38; A61K 33/34

(52) U.S. Cl. CPC A61K 9/0043; A61K 33/34; A61K 33/38; A61K 33/242

(57) ABSTRACT

A paper-thin, one-piece, reusable gender-conscious nasal congestion to mitigate the morbidity/mortality from airborne broad-spectrum pathogens with elemental gold nanosheets. Climate warming coupled with World population's explosion with increased population intra-inter-mobility will weigh on air-born infections and transmissions by an order of magnitude. Global demand for a gender-conscious nasal broad-spectrum biocide which is reusable and affordable, is estimated at three (3) billion dollars.

### FIELD

This invention relates to a nasal congestion comprising elemental nanosheets of gold as a prophylactic against broad-spectrum air-born bacteria and virus.

### BACKGROUND

The oligodynamic properties of metals were recognized since the era iron swords were applied to open-wounds by Roman soldiers. This invention uses these properties. Climate warming coupled with world populations' growth, increase the morbidity and mortality caused by air-born-pathogens by an order of magnitude. Furthermore, the world's voracious appetite for travel exacerbates global pandemics risks. The scientific community introduced antibiotics and “nanoparticles”, in an array of cumbersome nasal devices, but with little success and compliance across culture. The aforementioned “colloidal nanoparticles” of (Ag), copper (Cu), Nickel (Ni) became science's latest fashion and their nanoparticles were embedded in an array of patented inventions despite known carcinogen toxicity in direct proximate distance to the lungs, and despite nanoparticles limiting ionic lysis in contrast to the oligodynamic action enabled from wider metal-contact-surfaces. Conversely, this invention's ionic action of gold macro-surface physically impair intra-functions of both bacteria and virus, unlike antibiotics narrow effectiveness on bacteria. The elemental gold of this invention is a: (i) non-toxic neutral element. (ii) macro-wide-contact nanosheet for oligodynamic and quantum-wavelength biocides offering the best of two physics: a nano-thickness with macrowide-contact-surface. (iii) a mission critical gender-conscious look & feel to resolve collective avoidance of intrusive nasal congestions, with a simple device to encourage global market compliance.

### SUMMARY

The present invention features a treble asymmetric ionic attack. Despite gargantuan body of cell-research, understanding nano levels biofunctions remains elusive, especially for viral replication, the old model is incoherent as random route of exciton is flatly inefficient, and energy is hemorrhaged. Corollary, energy transfer may only be explained by a coherent pathways “wave” which explores all paths simultaneously and only take the most efficient route. Virus machinery might have evolved to exploit quantum phenomena, and we concur as virus otherwise could not have evolved. Processes at this scale are captured in the complex mathematical language of quantum mechanics, and frequently produce effects that appear to defy common sense. It is using this language that Vedral had detected signatures of entanglement between photons and bacteria in data from the Sheffield experiment in 2017. Here the invention replicates with 24K Gold the mirrors of Sheffield experiments and natural light's photons at the entrance of nasal cavities. It is posited that the gold ion particles may entangle behind the pathogens cell walls. Further research may be needed to ascertain this theory but a growing body of evidence is establishing quantum effects, and this invention is closest to create that inducive quantum entanglement.

In addition, the present invention relies on two other lysis processes. First it maximizes oligodynamic actions by optimizing two (2) elemental nano gold surface-area to an average of 1 cm2 a claim not made by any previous inventions, a distinction which simultaneously increase resonance in plasmonic transductions and has the critical benefit of increasing cross-gender compliance.

Which brings us to the mission critical exploration of optimum device's shapes to implement cross-gender compliance.

The fatal flaw of prior patented nasal congestions is ignoring our universal repulsion for inserting conspicuous devices up our own nasal cavities (women's aversion in studies across cultures were demonstrably 9 folds stronger than men). This critical oversight over clinical imperatives of acceptance and compliance renders futile prior patented inventions. By contrast this present invention most distinct feature is its Occam's razor simplicity and gender-conscious aesthetics, with a lightweight ≤0.07 g, imperceptible and transparent. The instantly recognizable iconic “ornaments” were proven to exhibit gender conscious positive connotations across cultures. The device features a layered spread of 24K elemental Gold with external transparency. These luxury connotations engendered consistent compliance in double blind meta-trials against all prior patented inventions.

Lastly, the world's appetite for travel being voracious, the demand for inter and intra travel counted (5) billion intercontinental airlines travelers and seventeen (17) billion land travelers in 2018. World's mobility coupled with global warming geometrically increase the risks of pandemics.

In sum: the device disclose the following characteristics, to wit:
(i) mitigate morbidity/mortality with improved biocidal properties;
(ii) low cost and ease of manufacturing for mass-production.
(iii) recyclability;
(iv) Look & feel gender-conscious-nasal-congestions designed for cross-cultural acceptance and compliance, are the hallmark of this present invention.

### BRIEF DESCRIPTION OF THE DRAWINGS

FIG. 1 Is a comparative overview of the Occam's razor shift-of-paradigm which contrast prior invented devices-versus a cross section of the instant 2D-one-piece invention.

FIG. 2 (a) Is a top view of the device's one-piece. A transparent planar substrate which comprising a left-and-right mirror image portion and a middle narrower connecting bridge.

FIG. 2 (b) Is a top view of the device's substrate comprising a mirror image of a left and a right portion covered with a layer of gold nanosheet, leaving the bridge transparent.

FIG. 2 (c) Is a cross-section view of the one-piece, paper-thin 2D device.

FIG. 2 (d) Is a cross-section of the one-piece, paper-thin 2D invention with a slight curvature of the connecting transparent bridge.

FIG. 2 (e) Is a top view of the array of various embodiments which the possible form of the mirror image of the left and right portion including the planar 2D and the folded planar 3D embodiments.

FIG. 3 (a) Is an exploded view of the device disclosing a method of applying the adhesive on the gold nanosheets to attach on the substrate.

FIG. 3 (b) Is an exploded view of the device disclosing a method of applying the adhesive on the substrate and then applying the gold nanosheets.

FIG. 4 (a) Is a top view an oblong shape of the 2D substrate prior of folding.

FIG. 4 (b) Is an isometric view of a folded substrate to form a spiral shape and the nanosheets applied on the outer surface.

FIG. 4 (c) Is an isometric view of a folded substrate to form a spiral shape and the nanosheets applied on the outer surface.

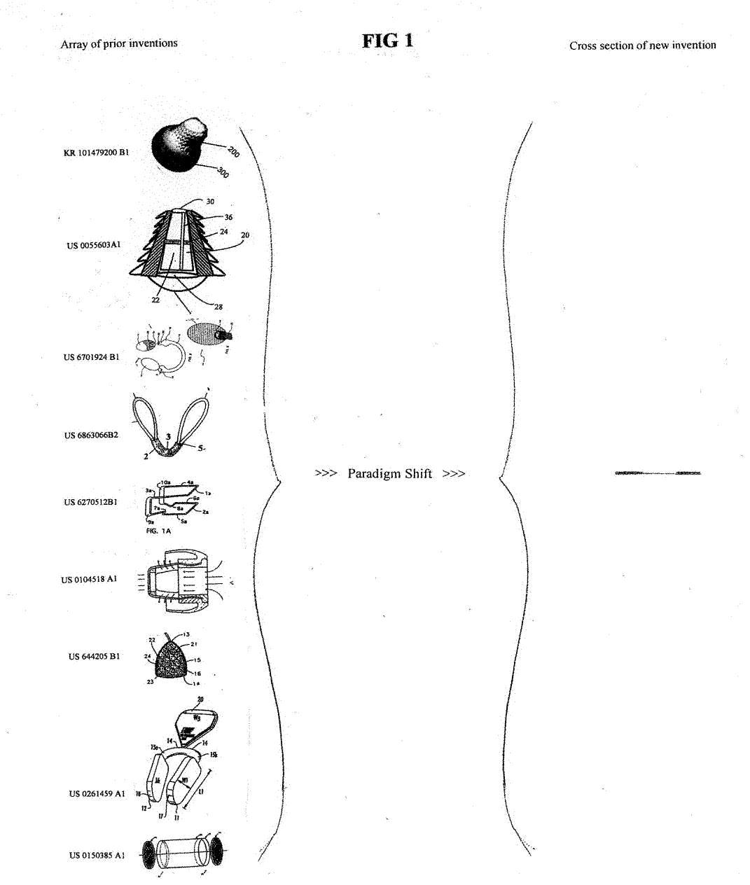

### DETAILED DESCRIPTION

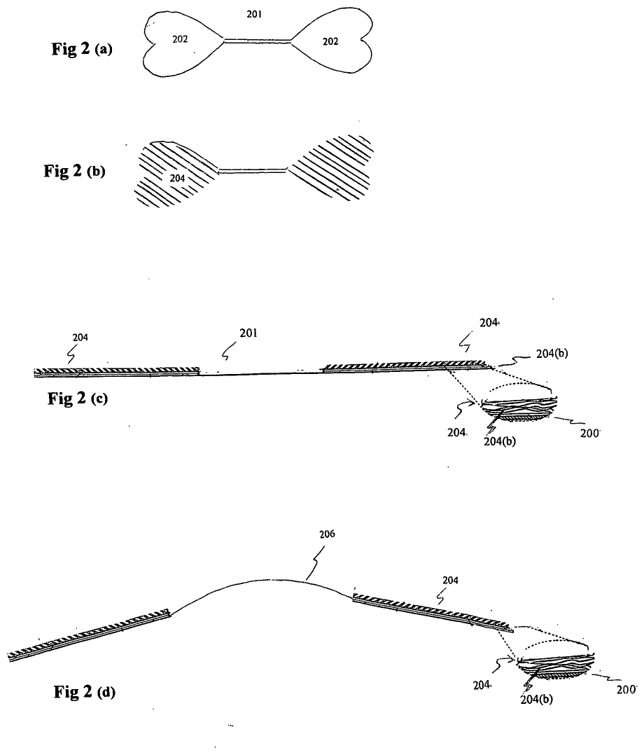

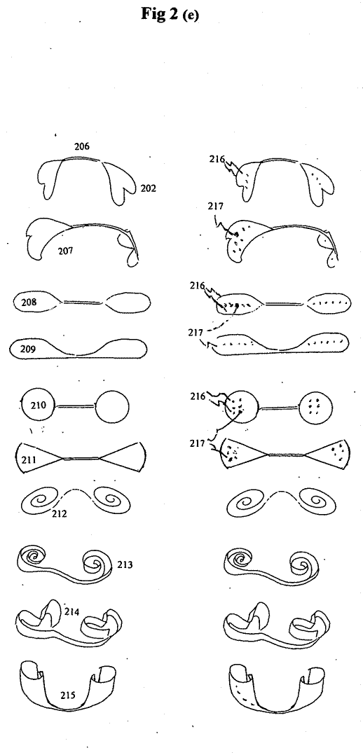

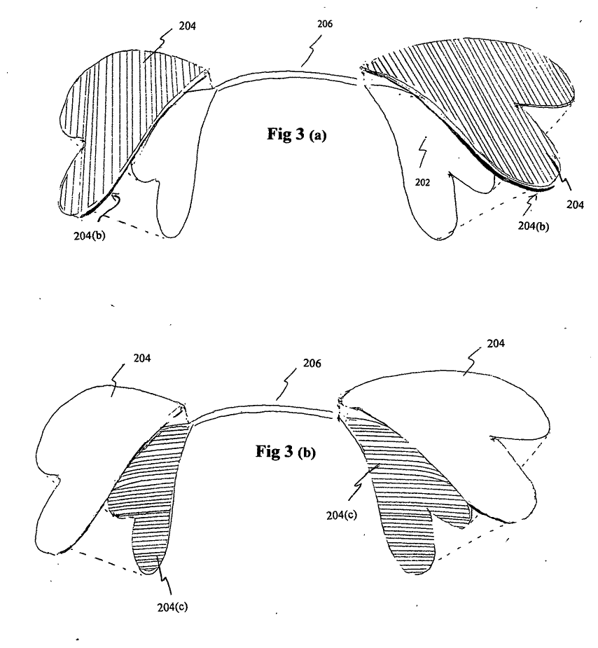

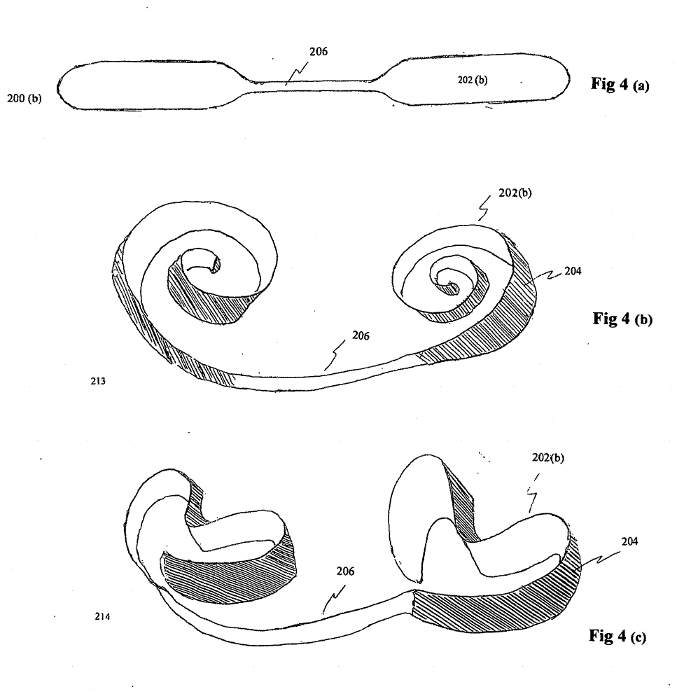

The present invention is a paper-thin transparent substrate made of a transparent sheet of polymers FIG. 2 (a). The device is a 2D fit one-piece transparent polymer frame which preferred embodiment has ˜≤0.5 mm in thickness, weighing ˜≤0.06 gr. Other embodiments could be thicker and/or heavier. The preferred device comprise a mirror image left and right portion (202) of the substrate which narrows itself to became a connective bridge in between (201). The total device is a one piece of a length of ˜4 cm≤range≤˜8 cm. The active broad spectrum biocidal (204) is a nanosheet of elemental Gold ≤24K of macro-wide surface 5 mm2≤range≤10 mm2 and a nano thickness of ˜30 nm≤range≤˜150 nm superimposing over the left and right mirror image portion of the polymer frame. The superimposition could also be partial in other embodiments. However, the bridge is left transparent/quasi-invisible. FIG. 2 (b) is a cross motion of the planar device that details the 3 superimposed layers to wit: (i) a polymer transparent substrate, (ii) an adhesive, (iii) the gold nanosheet. FIG. 2 (d) is a cross motion of the device with a curvature of the bridge. FIG. 2 (e) Is a top view of the array of various embodiments which the possible form of the mirror image of the left and right portion including the planar 2D and the folded planar 3D embodiments. The various alternative embodiments are herein listed. Drawing (206) is the 3D view of the curved bridge curbed longitudinally. The longitudinal curvature of the connecting transparent bridge is within a range ≤80° while the left and right portion display no curvature and remains flat, in another embodiment the curved bridge extend to a 3D concave curvature (207) of left and right portion of 80°≤range≤180°. Drawing (208) is the isometric view of a tear-shape-2D (208) device of bridge curvature Drawing (209) is the isometric view of a half-tear-shape-2D device. But with alternative embodiment of concave left and right portions. As for Drawing (210) is the isometric view of a round-shape-2D device with alternative concavity. Drawing (211)—is the isometric view of a 2D-conic-shape device with alternative embodiment of curvature and concavity similar to aforementioned devices. Drawing (212)—is the isometric view of a spiral-2D-shape device (212) according to a 16^{th} embodiment whereas the device is entirely a 2D planar geometry to be contrasted later with 3D spiral of FIG. 3, but both disclose a transparent bridge, the first worn flat under the nose the second worn flat on the philtrum, thus its entire width visible and a less preferred embodiment. Drawing (213)—is the isometric view of a spiral-2D-shape frame according to another embodiment whereas the frame itself is curled and rolled (213) constituting a 3D spiral allowing the external bridge be only visible cross-sectionally under the nose similar to all embodiments herein disclosed, thus invisible. (214)—is a heart-2D-frame according whereas the frame is folded or curled on itself constituting a 3D heart device (214) allowing the bridge be only cross-sectionally seen externally under flat the nose as to all the aforementioned drawings. Drawing (211) is the isometric view of a cylinder-2D-frame 19^{th} embodiment (215) whereas the frame is curled on itself to form a 3D cylinder allowing the bridge to be only cross-sectionally externally worn under the nose practically invisible. Drawing (216 and 217) have the same characteristics of the first 11 possible embodiments in addition to the following 3 enhancement features which can be implemented collectively to all. (i) a double-sided (216) layer of nanosheets 50 nm≤range≤150 nm (ii) the frame's left and right portion may have 3 to 6 pinholes (217) of 0.05 mm pinholes to increase airflow.

### General Characteristics and Manufacturing:

The simplicity of the invention's characteristics, to wit: the paper-thin 2D one-piece-unibody allows for affordable mass production. The present invention superimposition of the nanosheets is made with current cold stamping rolls on the polymer where the adhesive is already incorporate as a substrate to the stock roll goldsheets (204 b) as described in FIG. 2 (b); but also present a novel rapid adhesive method FIG. 2 (c) for adhering the nano gold spread onto a transparent polymer for ≤3 seconds adhesion with a substrate of 1 part latex and 19.7 parts C3H6O Dimethyl Ketone sprayed or brushed on flat polymer sheets followed by laser dye cutting of flat web-stock transparent soft polymer as disclosed in (204 c). The choice of the transparent polymer may be; cellulose acetate, polyethylene, acrylonitrile-butadiene-styrene (ABS), and a matrix of Trithiocarbonate PniPaam-based or poly-propylene, poly-ethylene terephthalate PET or any latest scientific improvement such as a responsive polymer as long as they are transparent and flexible substrate. However during a pandemic the polymer frame may be substituted by a 16-point<range<12-point cardstock. Followed by nanosheet gold application and then laser or stamping dye cut in the chosen shapes and in last stage dye heated for those convex device in different embodiments. The aforementioned designs that are laser/dye-cut have been selected specifically for gender-conscious popular design for positive symbolic conations of warmth and healthiness across cultures of spirals, tears, hearts oblongs, and concentric circles subsequently layered with nanosheet Au≤24KT Minding the toxicity of alloys Cu, Ni, Ag or mercury or similar metals the invention may use other metals during pandemics weighing risk of toxicity with morbidity/mortality in an epidemic. Another preferred embodiment is a double-sided gold layers. Another possible embodiment is rapid microwave exfoliation to ≤30 nm with the use of 150 C graphene dielectric H2O at 6 sec intervals.

What is claimed:

1. A nasal congestion comprising a substrate having a left/right mirror-image portions covered by a gold nanosheet layer and a middle bridge connecting them.

2. The nasal congestion of claim 1, wherein the macro-wide surface of the elemental gold nanosheet is 5 mm₂≤range≤10 mm₂ and of a micro-width of 30 nm≤range≤150 nm.

3. The nasal congestion of claim 1, wherein substrate is made of a transparent soft material.

4. The nasal congestion of claim 1, wherein substrate is 2D paper thin and made of one piece.

5. A device according to claim 1, wherein the left and right portion are covered with a metal nanosheet of preferred elemental gold or alternatively nanosheets of silver, copper or brass while the center connective bridge, external to the nasal cavity remains transparent.

6. The nasal congestion of claim 5, wherein the bridge part which will be externally tucked under the nose is quasi-invisible due to its 2D˜≤0.5 mm in thickness and transparency.

7. The nasal congestion according to claim 1, wherein it is disposable or washable and/or reusable.

8. The nasal congestion according to claim 1, wherein the 2D frame is mass produced by cutting from a flat transparent sheet of web-stock soft polymer by laser or stamp dye cut.

9. The nasal congestion of claim 3, wherein the look & feel for a gender-conscious device is created with gender-conscious forms for the left and right portion.

10. The nasal congestion of claim 8, wherein the look & feel for a gender-conscious device is created with gender-conscious iconic forms such as hearts, or tears, or spirals, or circles, or oblong designed for the left and right portion of the frame.

11. The nasal congestion of claim 1, in another embodiment wherein the connective bridge has a curvature and the left and right design portion are concave to form a 3D device from its 2D frame.

12. The nasal congestion of claim 11, wherein another embodiment folds the left/right portions fold to form a 3D heart or spiral shape or any similar 3D shape.

13. The nasal congestion of claim 1, wherein the nanosheets cover front-and-back sides of the left/right portions of the frame.

14. A device according to claim 5, of another embodiment for swift mass production during a pandemic wherein the active biocide may be substituted with Ag, Ni, Cu or a combination alloy over a frame of <14-point cardstock paper.

15. A manufacturing process for applying gold onto the polymer by adhesion either by utilizing commercial stamping (hot or cold) from gold rolls, an end product sold as a 130 nm nanosheet or further thinned by standard rapid microwave exfoliation to 30 nm. Another manufacturing process applies adhesive on the polymer frame and then apply the gold nanosheet overlay.
